(19) United States
(12) Patent Application Publication
(10) Pub. No.: US 2020/0375983 A1
(43) Pub. Date: Dec. 3, 2020

(54) A PROCESS FOR THE PREPARATION OF CARIPRAZINE HYDROCHLORIDE

(71) Applicant: AUROBINDO PHARMA LIMITED, Hyderabad (IN)

(72) Inventors: Praveen Kumar NEELA, Hyderabad (IN); Guruswamy BATTHINI, Hyderabad (IN); Sampath Kumar SANKINENI, Hyderabad (IN); Hariprasad Reddy BINGI, Hyderabad (IN); Raghavender Reddy MADDI, Hyderabad (IN); Sivakumaran MEENAKSHISUNDERAM, Hyderabad (IN)

(73) Assignee: Aurobindo Pharma Ltd, Hyderabad (IN)

(21) Appl. No.: 16/767,102

(22) PCT Filed: Nov. 21, 2018
(86) PCT No.: PCT/IB2018/059170

§ 371 Date: May 26, 2020

(30) Foreign Application Priority Data
Dec. 1, 2017 (IN) 201741043174

(51) Int. Cl. A61K 31/495

(52) U.S. Cl. CPC A61K 31/495

(57) ABSTRACT

The present invention is directed towards a process for the preparation of Cariprazine (Ia) or a pharmaceutically acceptable salt thereof, wherein, N,N-dimethyl-1H-imidazole-1-carboxamide alkyl halide (VII) is reacted with trans-4-(2-(4-(2,3-dichlorophenyl)piperazin-1-yl)ethyl)cyclohexanamine or a salt thereof (IIa) to produce Cariprazine (Ia) or a pharmaceutically acceptable salt thereof. The present invention is also directed towards amorphous form of Cariprazine hydrochloride (I) and solid dispersion of Cariprazine hydrochloride (I), and its preparation thereof.

### FIELD OF INVENTION

The present invention relates to an amorphous form of Cariprazine hydrochloride (I), or a solid dispersion thereof.

The present invention relates to a process for the preparation of Cariprazine hydrochloride (I).

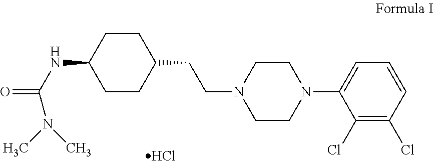

### BACKGROUND OF THE INVENTION

Cariprazine hydrochloride (I) is chemically known as trans-N-{4-[2-[4-(2,3-dichlorophenyl)piperazine-1-yl]ethyl]cyclohexyl}-N′,N′-dimethylurea hydrochloride salt. Cariprazine hydrochloride (I) is an atypical antipsychotic. Cariprazine hydrochloride (I) is being marketed in the US under the brand name Vraylar®.

Cariprazine (Ia) is disclosed in the U.S. Pat. No. 7,737,142.

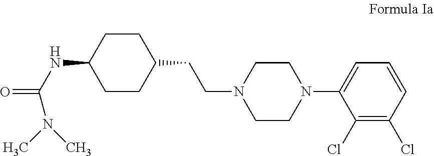

US '142, discloses a process for the preparation of Cariprazine (Ia), by reacting trans-4-(2-(4-(2,3-dichlorophenyl)piperazin-1-yl)ethyl)cyclohexanamine trihydrochloride (II) with N,N-dimethylcarbarnoylchloride (III) in presence of dichloromethane and triethylamine.

The Process is as Shown in Scheme-I Below:

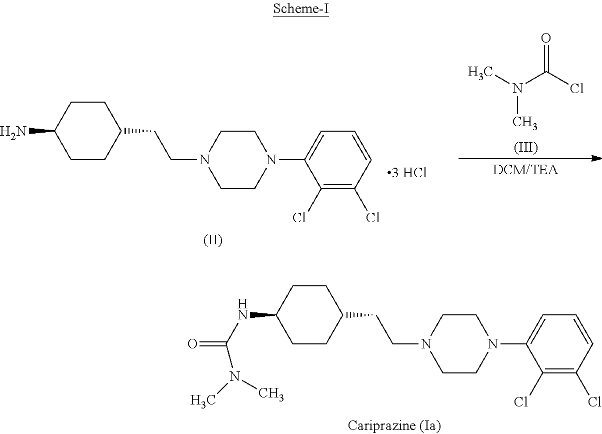

The disadvantage of the above process is the use of dimethyl carbamoyl chloride at final stage of the process is not suggestible due to its high toxicity and probable cancerogenicity in nature. The reaction is very slow thus time consuming process and also low yielding.

US '142 also discloses another process for the preparation of Cariprazine (Ia), by reacting trans-4-(2-(4-(2,3-dichlorophenyl)piperazin-1-yl)ethyl) cyclohexanamine trihydrochloride(II) with triphosgene in presence of dichloromethane and triethylamine to produce Isocyanate compound (IV), Which is further reacted with dimethylamine hydrochloride (V) to produce Cariprazine (Ia).

The Process is as Shown in Scheme-II Below:

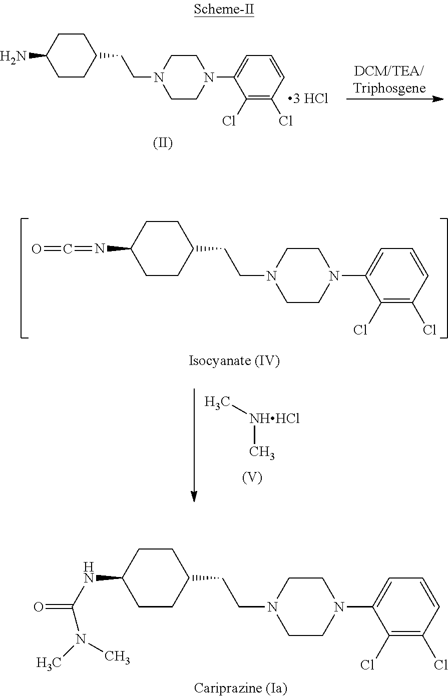

The disadvantage of the above process is the use of triphosgene at commercial scale may not be feasible because of its high toxicity or one of its precursors in order to prepare isocyanate (IV).

U.S. Pat. No. 9,718,795 discloses a process for the preparation of Cariprazine (Ia), by reacting N-[4-[2-[4-(2,3-dichlorophenyl)piperazin-1-yl]ethyl]cyclohexyl]-imidazol-1-carboxamide (VI) with N,N-dimethylamine or a salt thereof (Va).

The Process is as Shown in Scheme-III Below:

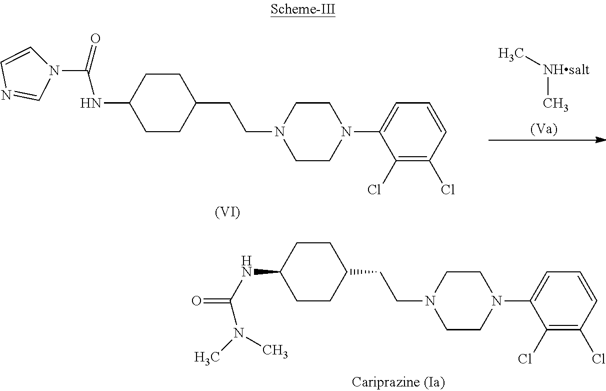

The disadvantage of the above process is the use of carbondimidazole (CDI) for amine activation. The use of carbondimidazole in commercial scale is limited and will not give reproducible results. It is highly unstable to even traces of moisture.

U.S. Pat. No. 7,943,621 discloses crystalline Form-I of Cariprazine hydrochloride (I) having a powder X-ray diffraction pattern that comprises peaks at about 6.6, about 7.3, about 13.2, about 21.1, and about 22.4, each about ±0.2° 2θ. Cariprazine hydrochloride (I) Form-I is prepared by adding Cariprazine (Ia) to a solvent or mixture of solvents followed by adding hydrochloric acid, or a salt thereof prepared from a base which is weaker base than the Cariprazine (Ia).

U.S. Pat. No. 7,829,569 discloses crystalline Form-III of Cariprazine hydrochloride (I) having a X-ray powder diffraction pattern comprising characteristic peaks at about 4.1, about 12.3, about 16.5, and about 17.4±0.2° 2θ. Cariprazine hydrochloride (I) Form-III is prepared by adding Cariprazine hydrochloride (I) to pyridine.

There remains a need for alternate form(s) of Cariprazine hydrochloride (I). Particularly, an amorphous form of a drug may exhibit a higher bioavailability than its crystalline counter parts, which leads to the selection of the amorphous form as the final drug substance for pharmaceutical dosage form development. Additionally, the solubility of crystalline form is lower than its amorphous form in some instances, particularly aqueous solubility, which may result in the difference in their in-vivo bioavailability. Therefore, it is desirable to have an amorphous form of a drug to meet the needs of drug development. Hence, it is desirable to provide an amorphous Cariprazine hydrochloride (I), optionally the amorphous form is in the form of a solid dispersion.

The present invention avoids the use of dimethyl carbonyl chloride in the carbonylation step and involves less reaction time; results high yield of the product, thus the process is economical and industrially viable. However, there is always a need for an alternate process, which for example, involves use of reagents that are less expensive and/or easier to handle, consume smaller amounts of reagents, provide a higher yield of product, have smaller and/or more eco-friendly waste products, and/or provide a product of higher purity.

Hence, there is a need to develop cost effective and commercially viable process for the preparation of Cariprazine (Ia).

### OBJECTIVE OF INVENTION

The main objective of the present invention is to provide an amorphous form of Cariprazine hydrochloride (I), optionally in the form of a solid dispersion.

The present invention also provide a simple, industrially feasible and cost effective process for the preparation of Cariprazine hydrochloride (I) with high purity and good yield on commercial scale.

### SUMMARY OF THE INVENTION

The main embodiment of the present invention is to provide Cariprazine hydrochloride (I) in an amorphous form, optionally in the form of a solid dispersion.

In another embodiment of the present invention is to provide a process for the preparation of Cariprazine hydrochloride (I) in an amorphous solid dispersion form comprising the steps of:
a) providing Cariprazine hydrochloride (I) solution;
b) adding a pharmaceutically acceptable polymeric material;
c) removing the solvent,
d) isolating amorphous solid dispersion of Cariprazine hydrochloride (I).

In another embodiment of the present invention is to provide a process for the preparation of Cariprazine hydrochloride (I) in an amorphous form comprising the steps of:
a) providing Cariprazine hydrochloride (I) solution;
b) removing the solvent,
c) isolating amorphous form of Cariprazine hydrochloride (I).

In another embodiment of the present invention is to provide a process for the preparation of Cariprazine hydrochloride (I) in an amorphous form comprising the steps of:
a) providing Cariprazine hydrochloride (I) solution;
b) adding an anti-solvent to precipitate Cariprazine hydrochloride (I);
c) isolating amorphous form of Cariprazine hydrochloride (I).

In another embodiment of the present invention is to provide a process for the preparation of Cariprazine (Ia) or a pharmaceutically acceptable salt thereof.

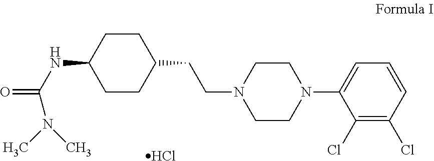

which comprises, reacting N,N-dimethyl-1H-imidazole-1-carboxamide alkyl halide (VII);

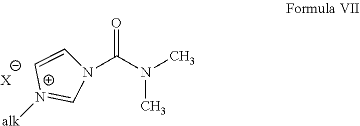

wherein X is a halide,
with trans-4-(2-(4-(2,3-dichlorophenyl)piperazin-1-yl)ethyl)cyclo hexanamine or a salt thereof (IIa)

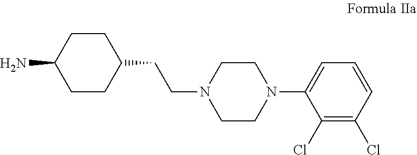

to produce Cariprazine (Ia) or a pharmaceutically acceptable salt thereof.

Yet another embodiment of the present invention is to provide a pharmaceutical composition comprising the use of amorphous form of Cariprazine hydrochloride (I) or a solid dispersion thereof, and one or more pharmaceutically acceptable excipients.

### BRIEF DESCRIPTION OF THE DRAWINGS

FIG. 1 Illustrates the X-ray powder diffraction pattern of Amorphous Cariprazine hydrochloride (I) produced by the present invention.

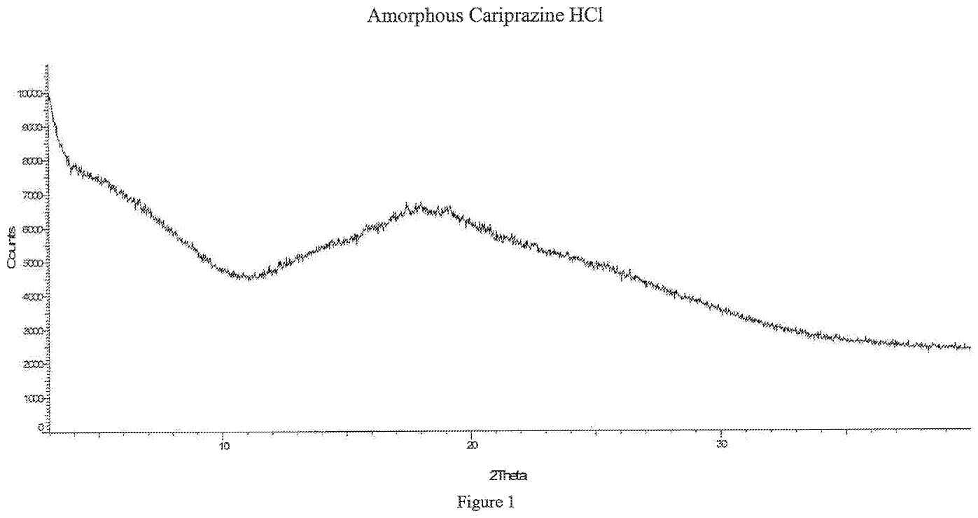

FIG. 2 Illustrates the X-ray powder diffraction pattern of crystalline Cariprazine (Ia) free base produced by the present invention.

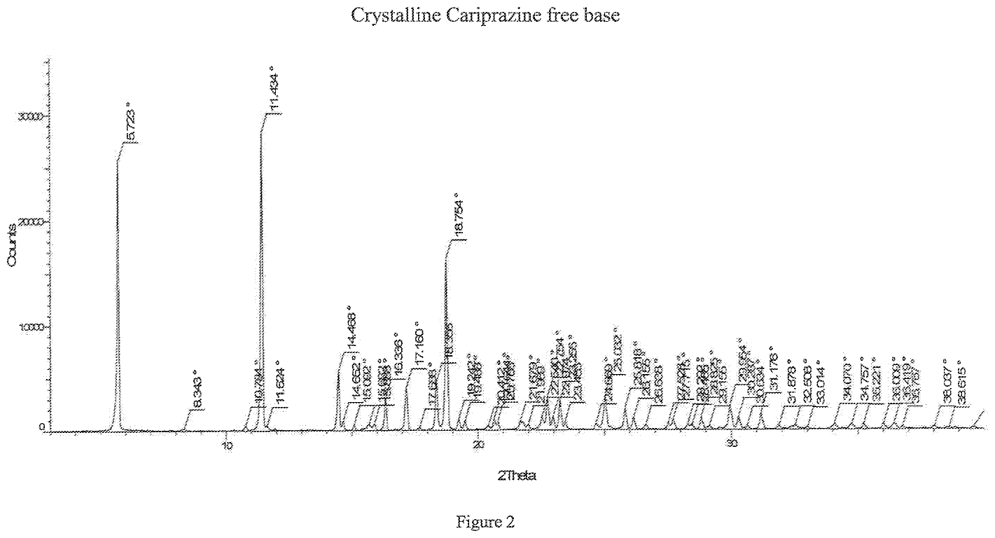

FIG. 3 Illustrates the X-ray powder diffraction pattern of Solid dispersion of Amorphous Cariprazine hydrochloride (I) produced by the present invention.

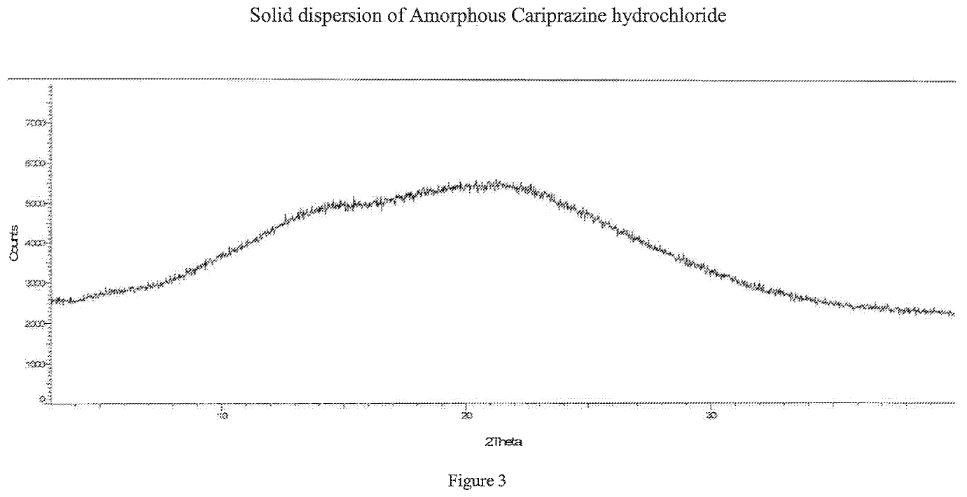

### DETAILED DESCRIPTION OF THE INVENTION

The present invention provides Cariprazine hydrochloride (I) in an amorphous form.

Yet another embodiment of the present invention provides a process for the preparation of Cariprazine hydrochloride (I) in an amorphous form.

The process comprises, providing Cariprazine hydrochloride (I) solution, which is subjected to removal of solvent and isolation to produce amorphous form of Cariprazine hydrochloride (I).

The solvent used in the process comprises water, ethanol, methanol propanol, isopropanol, butanol, acetone, methyl ethyl ketone, methyl isobutyl ketone, ethyl acetate, diethyl carbonate, methyl acetate, isobutyl acetate, diethyl ether, diisopropyl ether, 1,4-dioxane, methyl tert-butyl ether, tetrahydrofuran, methyltetrahydrofuran, methylene chloride, Carbon tetrachloride, chloroform, chloro benzene, benzene, toluene, hexane, cyclohexane, pentane, xylene, acetonitrile, dimethylformamide, dimethylsulfoxide or mixture thereof.

The removal of solvent is carried out by drying methods comprises air drying, spray drying, evaporation, distillation, oven drying, tray drying, rotavapor technique, freeze drying, fluid bed drying, flash drying, agitated thin film drying or melt extrusion method.

Another embodiment of the present invention provides a process for the preparation of Cariprazine hydrochloride (I) in an amorphous form, comprises providing Cariprazine hydrochloride (I) solution, followed by adding an anti-solvent to precipitate Cariprazine hydrochloride (I) and amorphous form of Cariprazine hydrochloride (I) is isolated.

The solvent used in the process comprises water, ethanol, methanol propanol, isopropanol, butanol, acetone, methyl ethyl ketone, methyl isobutyl ketone, ethyl acetate, diethyl carbonate, methyl acetate, isobutyl acetate, diethyl ether, diisopropyl ether, 1,4-dioxane, methyl tert-butyl ether, tetrahydrofuran, methyltetrahydrofuran, methylene chloride, carbon tetrachloride, chloroform, chloro benzene, benzene, toluene, hexane, cyclohexane, pentane, xylene, acetonitrile, dimethylformamide, dimethylsulfoxide or mixture thereof.

The anti-solvent used in the process comprises water, ethanol, methanol propanol, isopropanol, butanol, ethyl acetate, diethyl carbonate, methyl acetate, isobutyl acetate, benzene, toluene, hexane, cyclohexane, pentane or mixture thereof.

Yet another embodiment of the present invention provides the amorphous form of Cariprazine hydrochloride (I) is in the form of a solid dispersion.

Another embodiment of the present invention provides a process for the preparation of the amorphous form of Cariprazine hydrochloride (I) is in the form of a solid dispersion.

The process comprises, providing Cariprazine hydrochloride (I) solution and a pharmaceutically acceptable polymeric material is added, then subjected to removal of solvent and isolation of amorphous form of Cariprazine hydrochloride (I) in the form of solid dispersion.

The solvent used in above process comprises water, ethanol, methanol propanol, isopropanol, butanol, acetone, methyl ethyl ketone, methyl isobutyl ketone, ethyl acetate, Diethyl carbonate, methyl acetate, isobutyl acetate, diethyl ether, diisopropyl ether, 1,4-dioxane, methyl tert-butyl ether, tetrahydrofuran, methyltetrahydrofuran, methylene chloride, Carbon tetrachloride, chloroform, chloro benzene, benzene, toluene, hexane, cyclohexane, pentane, xylene, acetonitrile, dimethylformamide, dimethylsulfoxide or mixture thereof.

The polymeric material uses in the process comprises copovidone, povidone, hypromellose, hydroxylpropyl cellulose, polyvinyl caprolactam-polyvinyl acetate-polyethylene glycol, and the like.

The removal of solvent is carried out by spray drying, evaporation, distillation, oven drying, tray drying, rotavapor technique, freeze drying, fluid bed drying, flash drying, agitated thin film drying or melt extrusion method.

Yet another embodiment of the present invention is to provide a pharmaceutical composition comprising the use of amorphous form of Cariprazine hydrochloride (I) or a solid dispersion thereof, and one or more pharmaceutically acceptable excipients.

Another embodiment of the present invention is related to a process for the preparation of Cariprazine (Ia) or a pharmaceutically acceptable salt thereof.

The process comprises, N,N-dimethyl-1H-imidazole-1-carboxamide alkyl halide (VII) is reacted with trans-4-(2-(4-(2,3-dichlorophenyl) piperazin-1-yl)ethyl) cyclohexanamine or a salt thereof (IIa) to produce Cariprazine (Ia) or a pharmaceutically acceptable salt thereof.

The above reaction is carried out in the presence of a base and in the presence/absence of a solvent or mixture of solvents thereof. The base comprises an organic base selected from triethylamine, pyridine, methyl amine and 2,6-Lutidine or a inorganic base selected from potassium methoxide, potassium ethoxide, potassium tertiary butoxide, sodium methoxide, sodium ethoxide, sodium tertiary butoxide, sodium hydroxide, potassium hydroxide, lithium hydroxide, sodium carbonate, potassium carbonate, sodium bicarbonate, potassium bicarbonate, sodium acetate, potassium acetate or mixtures thereof. The solvent comprises methanol, ethanol, propanol, isopropanol, butanol, isobutanol, toluene, benzene, o-xylene, m-xylene, p-xylene, acetone, acetonitrile, ethyl acetate, methylene chloride, chloroform, dioxane, dimethylformamide, dimethyl sulfoxide, tetrahydrofuran, methyl tert-butyl ether, diethyl ether, hexane, cyclohexane, heptanes or mixture thereof.

The Cariprazine (Ia) is isolated as a solid or as such used in next step. Optionally, Cariprazine (Ia) is subjected to purification either by column chromatography or by crystallization by dissolving in a solvent or by adding an anti-solvent.

Cariprazine base (Ia) is treated with hydrochloride in a solvent to produce Cariprazine hydrochloride (I).

The solvent used in the above process comprises water, methanol, ethanol, propanol, isopropanol, butanol, isobutanol, toluene, benzene, o-xylene, m-xylene, p-xylene, acetone, acetonitrile, ethyl acetate, methylene chloride, chloroform, dioxane, dimethylformamide, dimethyl sulfoxide, tetrahydrofuran, methyl tert-butyl ether, diethyl ether, hexane, cyclohexane, heptanes or mixture thereof.

Cariprazine hydrochloride (I) is isolated as a solid crystalline form or as such used in the next step. Optionally, Cariprazine hydrochloride (I) is subjected to purification either by column chromatography or by crystallization by dissolving in a solvent or by adding an anti-solvent.

N,N-dimethyl-1H-imidazole-1-carboxamide alkyl halide (VII) used in the present invention is prepared by known methods or by a process comprises, reacting imidazole with dimethylcarbamic halide (VIII) to produce N,N-dimethyl-1H-imidazole-1-carboxamide (IX). The compound (IX) is reacted with an alkyl halide to produce N,N-dimethyl-1H-imidazole-1-carboxamide alkyl halide (VII);

The Process is as Shown in Scheme-IV Below:

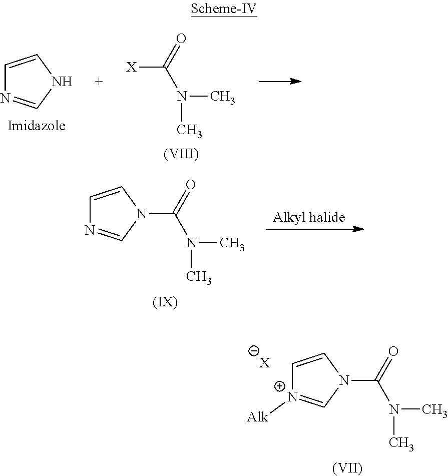

Wherein, the alkyl group is selected from C₁₋₄ carbon containing straight chain or substituted chain.

Trans-4-(2-(4-(2,3-dichlorophenyl)piperazin-1-yl)ethyl)cyclohexanamine or a salt thereof (IIa) used in the present invention is prepared by known methods or by a process comprises: protecting ethyl 2-(trans-4-amino cyclohexyl) acetate or a salt thereof (X) to produce N-protected ethyl 2-((1r,4r)-4-amino cyclohexyl) acetate (XI), which is reduced to provide N-protected 2-((1r,4r)-4-amino cyclohexyl) acetaldehyde (XII). The compound (XII) undergoes reductive amination with 1-(2,3-dichloro phenyl)piperazine or a salt thereof (XIII), followed by deprotection of resulted compound (XIV) to produce compound (IIa).

The Process is as Shown in Scheme-V Below:

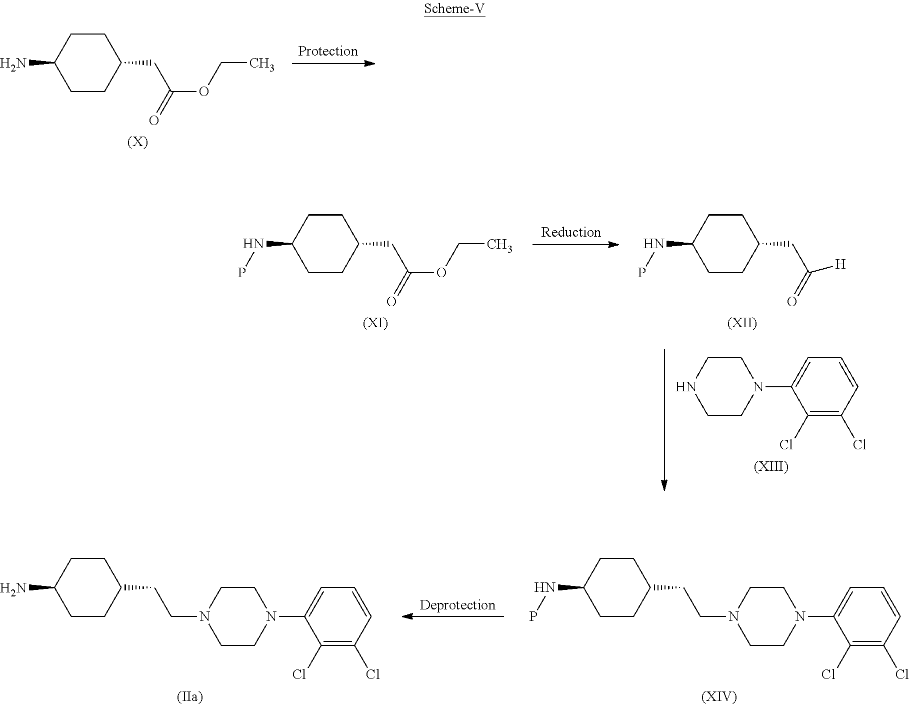

wherein P is protecting group.

The following example(s) illustrate the nature of the invention and are provided for illustrative purposes only and should not be construed to limit the scope of the invention.

### Example-1: Process for the Preparation of Cariprazine (Ia)

Trans-4-(2-(4-(2,3-dichlorophenyl)piperazin-1-yl)ethyl) cyclohexanamine (IIa) (100 g) was taken in to methylene chloride (2000 ml) and added triethylamine (71 g) followed by N,N-dimethyl-1H-imidazole-1-carboxamide methyl iodide (118.32 g) at room temperature. Tetra butyl ammonium bromide (9 g) was charged and the reaction mass was maintained for 6 hours at room temperature, and progress of reaction was monitored by HPLC. After completion of reaction, the reaction mass was filtered to remove undissolved salts and undissolved salt washed with methylene chloride. Filtrate was washed with water followed by sodium carbonate solution. Separated organic layer was treated carbon and distilled under reduced pressure, ethyl acetate and methanol was added to resulted residue and heated to 60-65° C. and maintained for 1.0 hr. The reaction mass was cooled to room temperature and stirred for 1.0 hr. The precipitated compound was filtered, washed with ethyl acetate and dried at 65° C.

Weight: 90 g; Yield: 75.07%.

Melting point: 196-200° C.

Cariprazine (Ia) prepared from above process characterized by following 20 values: 5.7±0.2 2θ, 11.4±0.2 2θ, 14.4±0.2 2θ, 15.8±0.2 2θ, 16.3±0.2 2θ, 17.1±0.2 2θ, 17.6±0.2 2θ, 18.7±0.2 2θ, 19.2±0.2 2θ, 22.9±0.2 2θ, 24.0±0.2 2θ, 25.8±0.2 2θ.

### Example-2: Process for the Preparation of Cariprazine Hydrochloride (I) from Cariprazine (Ia)

Cariprazine (Ia) (100 g) was dissolved in methylene chloride (3000 mL) and filtered through hyflow bed for removal of any particles and hyflow bed was washed with methylene chloride (200 ml). The filtrate was distilled under reduced pressure and methanol (200 ml) and water (820 ml) was charged to obtained residue at room temperature. Hydrochloric acid (22.9 ml) mixed with water (22 ml) was added at room temperature and stirred for 1 hr. The reaction mass was cooled to 0-10° C. and stirred for 5 hrs at same temperature. The precipitated compound was filtered, washed with water and dried at 70° C. under reduced pressure.

Weight: 97 g Yield: 89.36%.

### Example-3: Process for the Preparation of Amorphous Cariprazine Hydrochloride from Cariprazine Hydrochloride (I)

Cariprazine hydrochloride (I) (10 g) dissolved in methanol (200 mL) and the clear solution was passed into spray drier (Model: Buchi mini spray drier: B-290) at drier temperature 90° C., aspirator temperature 70° C. under 10 Kg/m³ pump pressure with 5 mL per minute flow rate.

Weight: 8.6 g Yield: 86%

### Example-4: Process for the Preparation of Amorphous Cariprazine Hydrochloride (I) from Cariprazine (Ia)

Cariprazine (Ia) (100 g) was dissolved in mixture of dichloromethane (700 mL) and methanol (300 mL) and added hydrochloric acid dissolved in methanol (8.53 g), the clear solution was passed into spray drier (Model: Buchi mini spray drier: B-290) at drier temperature 90° C., aspirator temperature 70° C. under 10 Kg/m³ pump pressure with 5 mL per minute flow rate.

Weight: 88.4 g; Yield: 81.8%.

### Example-5: Preparation of a Solid Dispersion of Cariprazine Hydrochloride (I)

Methanol (2000 ml) was taken into a RB flask at 25-35° C. Cariprazine hydrochloride (I) (100 g) was charged followed by Povidone or Copovidone (100 g) was charged into reaction mass at 25-35° C. The reaction mass was stirred for 10 mins at 25-35° C. and filtered. The solution was passed into spray drier (Model: Buchi mini spray drier: B-290) at drier temperature 100° C., aspirator temperature 70° C. with 10 mL per minute flow rate. The compound was collected and dried under reduced pressure at 25-35° C.

We claim:

1. Cariprazine hydrochloride (I) in an amorphous solid dispersion form.

2. A process for the preparation of Cariprazine hydrochloride (I) in an amorphous solid dispersion form as claimed in claim 1, comprising the steps of:
a) providing Cariprazine hydrochloride (I) solution in a solvent;
b) adding a pharmaceutically acceptable polymeric material;
c) removing solvent,
d) isolating amorphous solid dispersion of Cariprazine hydrochloride (I).

3. (canceled)

4. (canceled)

5. The process of claim 2, wherein the polymeric material used in step (b) comprises copovidone, povidone, hypromellose, hydroxylpropyl cellulose, polyvinyl caprolactam-polyvinyl acetate-polyethylene glycol, and the like.

6. The process of claim 2, wherein the removal of solvent is carried out by drying methods comprises air drying, spray drying, evaporation, distillation, oven drying, tray drying, rota-vapor technique, freeze drying, fluid bed drying, flash drying, agitated thin film drying or melt extrusion method.

7. A process for the preparation of Cariprazine (Ia) or a pharmaceutically acceptable salt thereof. 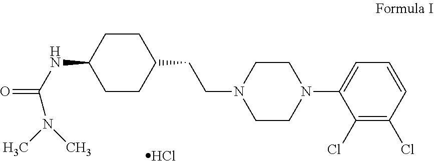
which comprises, reacting N,N-dimethyl-1H-imidazole-1-carboxamide alkyl halide (VII); 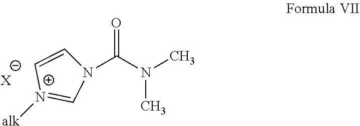
wherein X is a halide,
with trans-4-(2-(4-(2,3-dichlorophenyl)piperazin-1-yl)ethyl) cyclohexanamine or a salt thereof (IIa) 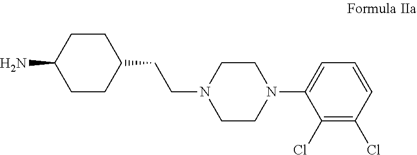
to produce Cariprazine (Ia) or a pharmaceutically acceptable salt thereof.

8. The process as claimed in claim 7, wherein the reaction is carried out in the presence of a base comprises an organic base selected from triethylamine, pyridine, methyl amine and 2,6-Lutidine or an inorganic base selected from potassium methoxide, potassium ethoxide, potassium tertiary butoxide, sodium methoxide, sodium ethoxide, sodium tertiary butoxide, sodium hydroxide, potassium hydroxide, lithium hydroxide, sodium carbonate, potassium carbonate, sodium bicarbonate, potassium bicarbonate, sodium acetate, potassium acetate or mixtures thereof.

9. The process as claimed in claim 2, wherein the solvent used in the process comprises water, ethanol, methanol propanol, isopropanol, butanol, acetone, methyl ethyl ketone, methyl isobutyl ketone, ethyl acetate, Diethyl carbonate, methyl acetate, isobutyl acetate, diethyl ether, diisopropyl ether, 1,4-dioxane, methyl tert-butyl ether, tetrahydrofuran, methyltetrahydrofuran, methylene chloride, Carbon tetrachloride, chloroform, chloro benzene, benzene, toluene, hexane, cyclohexane, pentane, xylene, acetonitrile, dimethylformamide, dimethylsulfoxide or mixture thereof.

10. A pharmaceutical composition comprising the use of amorphous form of Cariprazine hydrochloride (I) or a solid dispersion thereof as claimed in claim 1, and one or more pharmaceutically acceptable excipients.

11. The process as claimed in claim 5, wherein the solvent used in the process comprises water, ethanol, methanol propanol, isopropanol, butanol, acetone, methyl ethyl ketone, methyl isobutyl ketone, ethyl acetate, Diethyl carbonate, methyl acetate, isobutyl acetate, diethyl ether, diisopropyl ether, 1,4-dioxane, methyl tert-butyl ether, tetrahydrofuran, methyltetrahydrofuran, methylene chloride, Carbon tetrachloride, chloroform, chloro benzene, benzene, toluene, hexane, cyclohexane, pentane, xylene, acetonitrile, dimethylformamide, dimethylsulfoxide or mixture thereof.
